(19) United States
(12) Patent Application Publication
(10) Pub. No.: US 2017/0086667 A1
(43) Pub. Date: Mar. 30, 2017

(54) APPARATUS AND METHOD FOR WAVEFRONT GUIDED VISION CORRECTION

(71) Applicant: CLARITY MEDICAL SYSTEMS, INC., Pleasanton, CA (US)

(72) Inventors: YAN ZHOU, Pleasanton, CA (US); RUSSELL PON, San Ramon, CA (US); BARRY LINDER, Danville, CA (US)

(21) Appl. No.: 15/244,901

(22) Filed: Aug. 23, 2016

Related U.S. Application Data
(60) Provisional application No. 62/222,937, filed on Sep. 24, 2015.

(51) Int. Cl. A61B 3/10; G02B 13/00; G02B 27/00; G02B 27/10

(52) U.S. Cl. CPC A61B 3/1015; G02B 27/108; G02B 13/0095; G02B 27/0068

(57) ABSTRACT

Considered herein is a real time wavefront sensor and system to provide live feedback for various vision correction procedures such as LRI/AK refinement, Laser Enhancement, and cataract/refractive surgery, designed to reduce or eliminate interference during an examination and/or a surgical operation. Wavefront sensor modules attached to microscopes can reduce the available working space or working distance for an operator or surgeon. Use of a negative lens, rather than a non-lens optical shield or window, can increase in the working distance for an operator or surgeon to, at least in part, compensate for any reduction in the working space as a result of the attachment of the wavefront sensor module to the microscope.

### CROSS-REFERENCE TO RELATED APPLICATIONS

The present application claims benefit of priority to U.S. Provisional Application No. 62/222,937 entitled “APPARATUS AND METHOD FOR WAVEFRONT GUIDED VISION CORRECTION” and filed on Sep. 24, 2015, the entirety of which is incorporated by reference herein.

### TECHNICAL FIELD OF THE INVENTION

One or more embodiments of the present invention relate generally to wavefront sensors for determining the refractive state and/or wavefront aberrations of an eye and methods for wavefront guided vision correction.

### BACKGROUND OF THE INVENTION

Optical wavefront sensors are devices used to measure the shape of a wavefront of light (see, for example, U.S. Pat. No. 4,141,652 and U.S. Pat. No. 5,164,578). In most cases, a wavefront sensor measures the departure of a wavefront from a reference wavefront or an ideal wavefront such as a planar wavefront. A wavefront sensor can be used for measuring both low order and high order aberrations of various optical imaging systems such as the human eye (see for example, U.S. Pat. No. 6,595,642; J. Liang, et al. (1994) “Objective measurement of the wave aberrations of the human eye with the use of a Hartmann-Shack wave-front sensor,” J. Opt. Soc. Am. A 11, 1949-1957; T. Dave (2004) “Wavefront aberrometry Part 1: Current theories and concepts” Optometry Today, 2004 Nov. 19, page 41-45).

Furthermore, a wavefront sensor can also be used in adaptive optics in which the distorted wavefront can be measured and compensated in real time, using, for example, an optical wavefront compensation device such as a deformable mirror (see for example U.S. Pat. No. 6,890,076, U.S. Pat. No. 6,910,770 and U.S. Pat. No. 6,964,480). As a result of such compensation, a sharp image can be obtained (see, for example, U.S. Pat. No. 5,777,719).

Currently, most wavefront sensors for measuring the aberration of a human eye are bulky and are designed to only cover a limited diopter range of about −20D to +20D for a phakic or pseudo-phakic eye. In addition, they are also designed to operate in a relatively dark environment when the eye wavefront is to be measured. Furthermore, there is generally also a requirement for a patient eye and/or head to be positioned steady relative to the wavefront sensor for the wavefront measurement.

During ophthalmic refractive surgeries, it is desirable to know the refractive state of the eye as the surgery is on-going so that a continuous feedback can be provided to the surgeon (see for example, U.S. Pat. No. 6,793,654, U.S. Pat. No. 7,883,505, and U.S. Pat. No. 7,988,291). This is especially the case in cataract surgery in which the natural lens of the eye is replaced by an artificial synthetic lens called intra-ocular lens or IOL. In such a case, the surgeon prefers to know the refraction of the eye in the phakic, aphakic and pseudo-phakic stage in order to select a synthetic lens, confirm if the refractive power of the eye is as expected after the natural lens is removed, change the power of the synthetic lens if needed before implanting the synthetic lens, and also to confirm emmetropia or other intended diopter values such as −1D to −2D myopia after the synthetic lens is implanted. In addition, there is also a need to make the wavefront sensor immune to illumination light from a surgical microscope or room lighting. Furthermore, there also is a need for a compact wavefront sensor design so that it can be attached to or integrated with a surgical microscope to cover the needed larger diopter range and to measure the refraction of the eye with high enough accuracy and precision.

To address the issues, we have disclosed in co-assigned patents (see, for example, U.S. Pat. No. 7,445,335, U.S. Pat. No. 7,815,310, U.S. Pat. No. 8,100,530, U.S. Pat. No. 8,678,591, and U.S. Pat. No. 8,919,957) a lock-in detection based sequential wavefront sensor that can relay a large diopter range wavefront from a patient eye under a surgical microscope to a wavefront image plane via a relatively small beam scanner.

However, there still exists a need to further improve the performance of the wavefront sensor in terms of wavefront measurement accuracy and precision, to make the hardware of the apparatus more suitable for intra-operative wavefront sensing and real time guidance, and also to smartly use the wavefront sensor in optimizing the outcome of a refractive vision correction procedure.

### SUMMARY OF THE INVENTION

One or more embodiments of the present invention satisfy one or more of the above-identified needs in the art. In particular, one embodiment is the use of a lens rather than a non-lens optical shield or window at the input port of a wavefront sensor module to be attached to or integrated with an ophthalmic microscope. This lens is shared by the wavefront sensor module and the surgical microscope and can be used to flexibly provide a desired change in the working distance of the microscope for a surgeon. This lens can also be used to partially or fully compensate astigmatism that can be introduced to the microscope as a result of the use of a tilted main beam splitter and the tilting of the shield window itself, as well as to partially or fully compensate the astigmatism that can be introduced to the wavefront sensor as a result of the use of a slightly tilted or off-centered front lens of the wavefront relay configuration of the wavefront sensor module.

The tilting of the front lens of the wavefront relay is also by itself an aspect of the present disclosure as it can further reduce potential optical noise that may degrade the performance of the wavefront sensor. By slightly tilting or off-center positioning the front lens of the wavefront relay, the front-lens-caused specular reflections of a wavefront generating beam launched from behind the front lens can be directed away from the wavefront relay beam path so that they do not get channeled to the wavefront sensing detector.

Another embodiment is the use of at least two stacked dynamic focus variable lenses at or near a conjugate plane of an object wavefront within a wavefront relay to null or compensate (either partially or fully) the sphere component of the object wavefront from a patient eye to improve the accuracy and precision of wavefront measurement over a large diopter range.

Another embodiment is the use of at least one variable lens that can change both the sphere and the cylinder or astigmatism of a relayed wavefront at or near a conjugate plane of an object wavefront within a wavefront relay to null or compensate (either partially or fully) the sphere and/or cylinder/astigmatism component of the wavefront from a patient eye to further improve the accuracy and precision of wavefront measurement over a large diopter range.

Still another embodiment is the use of at least one cylinder or astigmatism variable lens combined with at least one focus variable lens at or near a conjugate plane of an object wavefront within a wavefront relay to null or compensate (either partially or fully) the cylinder/astigmatism and/or the sphere component of the wavefront from a patient eye to further improve the accuracy and precision of wavefront measurement over a large diopter range.

Still another embodiment is the use of a wavefront sampling aperture (as a spatial filter) with extremely absorptive coating on at least the side that faces the object to substantially absorb the portion of the relayed wavefront that is blocked by the wavefront sampling aperture so that minimum optical noise is generated from the aperture-blocked portion of the relayed wavefront.

Still another embodiment is to arrange a mega aperture at a first Fourier transform plane of the wavefront relay path of the wavefront sensor to serve the function of limiting the cone angle of the light rays from the eye and hence the diopter measurement range of the wavefront from the eye to a desired range as well as to prevent stray light from landing outside the movable mirror surface area of a beam scanner such as a MEMS scanner that is disposed at the second Fourier transform plane of the wavefront relay.

Furthermore, to simplify the self-calibration checking of the wavefront sensor, a dichroic beam splitter is combined with a reference wavefront generating light source such as a light emitting diode (LED) inside the wavefront sensor module to generate a reference wavefront for the wavefront sensor to measure. In addition, the self-generated reference wavefront light beam can also be made to travel through the focus and/or astigmatism variable lens(es) such that by changing the focus and/or the astigmatism of the focus and/or astigmatism variable lens(es), the response or transfer function of the wavefront sensor over a relatively large dioptric variation range can be checked.

In addition, to make sure that pointing direction of the SLD beam being sent to a patient eye is not moved over time, another embodiment of the present disclosure is to direct a very small amount of the SLED beam to an image sensor inside the wavefront sensor module for internal calibration and verification checking.

Still furthermore, in order to reduce to a minimum the disturbance to the normal flow of the procedure of a cataract or vision correction surgeon, one embodiment of the present disclosure is to automatically detect the state of the eye, especially when a patient eye is transformed from a phakic state to an aphakic state and then to a pseudo-phakic state.

Still another embodiment is to display, for each state of the patient eye, a qualified running time-averaged refraction or wavefront of the eye under operation that is most stable.

Still another embodiment is to automatically and/or continuously feed the time-averaged data obtained at the aphakic state of a patient eye into an IOL algorithm calculator.

Still another embodiment is to display the continuous result from the IOL algorithm calculator as a predicted IOL based on target refraction that is defaulted to emmetropia but can be manually entered pre-operatively if the target dioptric value is different from zero.

Still another embodiment is to automatically turn off the IOL calculator once the system detects that the surgical eye state is changed to pseudo-phakic.

These and other features and advantages of the example embodiments will become more readily apparent to those skilled in the art upon review of the following detailed description of the preferred embodiments taken in conjunction with the accompanying drawings.

### BRIEF DESCRIPTION OF THE DRAWING

FIG. 1 shows one schematic diagram of the presently disclosed wavefront sensor module and its attachment to a surgical microscope, according to embodiments of the present disclosure.

FIG. 1A shows three focus variable liquid lenses that are stacked together to function as a combined focus variable lens that can offer a large focusing power change, according to embodiments of the present disclosure.

FIG. 1B shows a diagram of a virtual image of a patient eye formed by a negative shield lens and the relative position of the virtual image with respect to the real patient eye, according to embodiments of the present disclosure.

FIG. 1C shows a light ray diagram, further illustrating how a negative lens bends the light rays to form a virtual image that appears closer to the negative lens when compared to the real object, according to embodiments of the present disclosure.

### DETAILED DESCRIPTION

Reference will now be made in detail to various embodiments of the invention. Examples of these embodiments are illustrated in the accompanying drawings. While the invention will be described in conjunction with these embodiments, it will be understood that it is not intended to limit the invention to any embodiment. On the contrary, it is intended to cover alternatives, modifications, and equivalents as may be included within the spirit and scope of the invention as defined by the appended claims. In the following description, numerous specific details are set forth in order to provide a thorough understanding of the various embodiments. However, the present invention may be practiced without some or all of these specific details. In other instances, well known process operations have not been described in detail in order not to unnecessarily obscure nor apply limitations to the present invention. Further, each appearance of the phrase an “example embodiment” at various places in the specification does not necessarily refer to the same example embodiment.

In recent years, it has been realized that there is a need for a real time wavefront sensor to provide live feedback for various vision correction procedures such as LRI/AK refinement, Laser Enhancement, and cataract/refractive surgery. For these procedures, any interference to a normal surgical operation is undesirable. Examples include the turning off of a surgical microscope's illumination light, the adjustment of the microscope height to a very limited range, the positioning of the patient eye in terms of its transverse position and tip/tile angle, and a waiting period for wavefront data capturing and processing. Surgeons want a real time feedback to be provided to them as the vision correction procedure is being normally performed.

Most prior art ophthalmic wavefront sensors for human eye wavefront measurements use a two dimensional CCD/CMOS image sensor for wavefront information collection. Due to the large amount of data that needs to be collected by the two dimensional image sensor and the limit in the frame rate resulting from the clock rate and/or the data transfer rate over an electronic data transfer line such as a USB cable, the image sensors used in these wavefront sensor devices generally operate with a relatively low frame rate (typically at 25 frames per second) and hence they are sensitive to DC or low frequency background noise.

Co-assigned U.S. Pat. No. 7,445,335 discloses a sequential wavefront sensor that sequentially shifts the entire wavefront to allow only a desired portion of the wavefront to pass through a wavefront sampling aperture. This wavefront sensor employs lock-in detection to reject DC or low frequency optical or electronic noise by pulsing at a relatively high repetition frequency the light source used for generating the wavefront from the eye and synchronizing it with a high frequency response position sensing device/detector. Therefore, this wavefront sensor does not require a dark environment for wavefront measurement, and is extremely suitable for continuous real time intra-operative refractive surgeries even with the illumination light of a surgical microscope in the “on” state. Another co-assigned U.S. patent (U.S. Pat. No. 8,356,900) discloses improved optical configurations over U.S. Pat. No. 7,445,335, allowing the use of a relatively small and commercially available light beam scanner (such as a MEMS scanner) to scan/shift the entire wavefront (over a large diopter measurement range) with respect to a wavefront sampling aperture disposed at a wavefront image plane. By flexibly scanning/shifting the wavefront, any portion of the wavefront can be sampled.

In another co-assigned U.S. patent (U.S. Pat. No. 9,039,181), it is disclosed that by disposing a variable focal length lens in a wavefront relay near a conjugate plane of an object wavefront, the sphere component of a relayed wavefront at a wavefront image plane can be dynamically changed. This is beneficial in terms of increasing the diopter measurement dynamic range because among the different wavefront aberration components, the sphere component generally has the largest diopter variation range among different eyes and also even for the same eye, it has the largest diopter variation range during a cataract surgery when the eye changes its state from phakia to aphakia and to pseudo-phakia. By dynamically nulling or reducing the sphere component (partially or fully), the drive signal sent to the variable focal length lens can be used to fully or partially provide the diopter value of the sphere component, and meanwhile the measurement precision and accuracy of the remaining wavefront aberration components can also be improved especially when a quadrant detector/sensor is used as a position sensing device. This is because compared to the sphere component, the diopter variation range of other wavefront aberration components such as the cylinder component and the coma component are much smaller and a quadrant detector/sensor functions the best when a light spot is close to its center, i.e. when the average tilt of a sampled sub-wavefront is small.

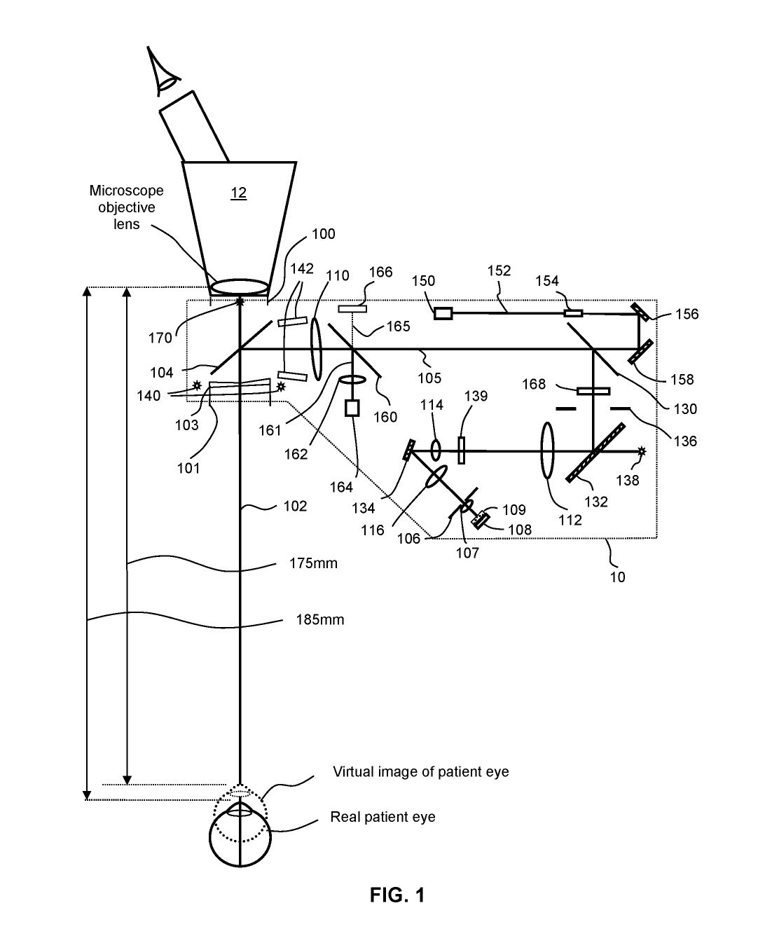

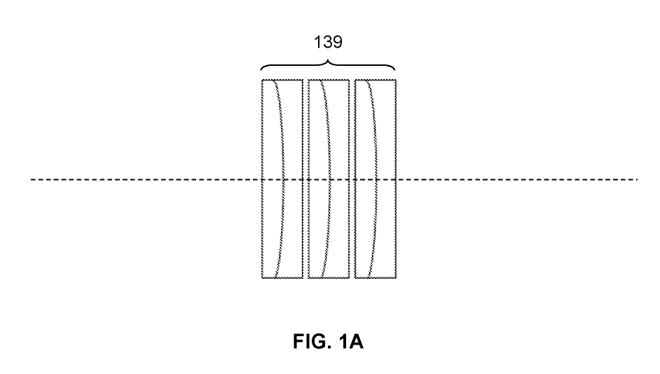

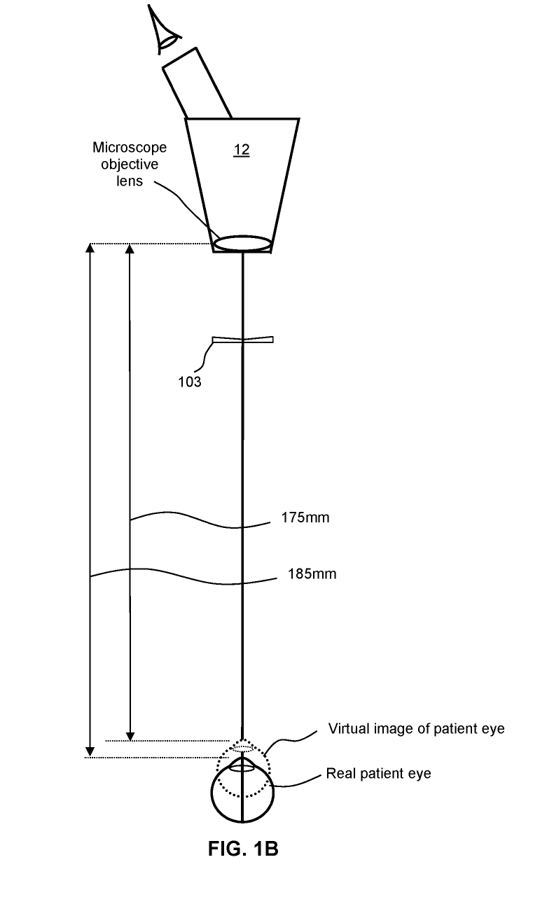

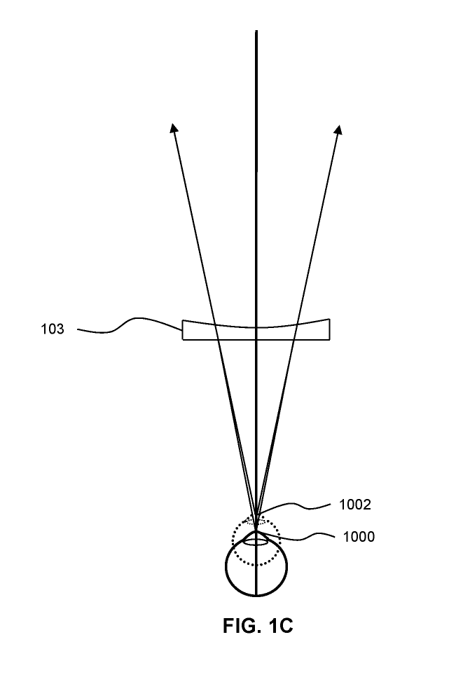

FIG. 1 shows one example embodiment of the optical configuration of a large diopter range real time sequential wavefront sensor module 10 attached to or integrated with a surgical microscope 12. The wavefront sensor module has an upper surface adjacent to the object lens of the surgical microscope 12 and a lower surface. First and second windows 100 and 101 are formed, respectively, in the upper and lower surfaces. The first and second optical windows 100 and 101 are aligned so that a first optical path 102 is formed between the first and second windows 100 and 101 that allows light returned from the patient eye to pass through the housing of the wavefront sensor module 10 to the objective lens of the surgical microscope. The first optical path 102 is aligned along the optical axis of the objective lens of the surgical microscope 12.

In the embodiment of FIG. 1, a lens shield 103 is disposed in the second optical window 101. The function of the lens shield 103 will be described in detail below.

A dichroic or short pass beam splitter 104 is disposed in the first optical path between the first and second windows and is used to reflect/deflect with high efficiency the near infrared wavefront relay beam (covering at least the optical spectral range of the superluminescent diode or SLD) to the rest of the wavefront sensor module while allowing most (for example ˜85%) of the visible light to pass through.

In the example embodiment depicted in FIG. 1, the dichroic or short pass beam splitter 104 reflects a near infrared wavefront beam as well as some imaging light along a wavefront relay beam path 105 interior to the wavefront sensor module 10. In this embodiment the wavefront relay beam path is folded using mirrors and beam splitters as described below.

In FIG. 1, the wavefront from the eye is relayed to a wavefront sampling image plane 8-F downstream at which a wavefront sampling aperture 106 is disposed. A sub-wavefront focusing lens 107 behind the aperture 106 focuses a sequentially sampled sub-wavefront onto a position sensing device/detector (PSD) 108 (such as a quadrant detector/sensor or a lateral effect position sensing detector). In this embodiment a diffuser 109 is positioned in front of the PSD 108. The wavefront relay is accomplished using two cascaded 4-F relay stages or an 8-F wavefront relay comprising a first lens 110, a second lens 112, a third lens 114, and a fourth lens 116.

The wavefront relay beam path 105 is folded by a polarization beam splitter (PBS) 130, a dichroic mirror 132 and a MEMS beam scanning/shifting/deflecting mirror 134 to make the wavefront sensor module compact. In addition, an aperture 136 can be arranged at the first Fourier transform plane between the PBS 130 and the dichroic mirror 132 to serve the function of limiting the cone angle of the light rays from the eye and hence the diopter measurement range of the wavefront from the eye to a desired range as well as to prevent light from landing outside the mirror surface area of the MEMS scanner 134. A reference wavefront generating light source 138 is located behind the dichroic mirror somewhere near the front focal plane of the second wavefront relay lens 112.

The MEMS scan mirror 134 is disposed at the second Fourier transform plane of the 8-F wavefront relay to angularly scan the object beam so that the relayed wavefront at the final wavefront image plane can be transversely shifted relative to the wavefront sampling aperture 106. The wavefront sampling aperture 106 can be a fixed size or an active variable aperture.

One requirement for real time ophthalmic wavefront sensor is a large diopter measurement dynamic range that can be encountered during a cataract surgery, such as when the natural eye lens is removed and the eye is aphakic. The dynamic measurement range of the wavefront sensor can be limited by the position sensing device/detector (PSD), especially in the case when a quadrant detector is used as the PSD. This is because if the overall tilt angle of a sampled sub-wavefront beamlet is relatively large, the corresponding light spot on the quadrant detector may land further away from the center of the quadrant detector and therefore miss one or two of the 4 quadrants, which will hence render the quadrant detector not being able to produce a reliable ratio-metric value to indicate the centroid position of the light spot.

In this embodiment, a dynamic wavefront/defocus offsetting device 139 is disposed at the intermediate wavefront image plane, i.e. the 4-F plane which is conjugate to both the corneal plane and the wavefront sampling plane.

The dynamic wavefront/defocus offsetting device 139 can be a drop-in lens, a focus variable lens, a liquid crystal based transmissive wavefront manipulator, or a deformable mirror based wavefront manipulator. In the case that the PSD becomes the limiting factor for measuring a large diopter value (positive or negative), the wavefront/defocus offsetting device 139 can be activated to offset or partially/fully compensate some or all of the wavefront aberrations. For example, in the aphakic state, the wavefront from the patient's eye is relatively divergent, a positive lens can be dropped into the wavefront relay beam path at the 4-F wavefront image plane to offset the spherical defocus component of the wavefront and therefore to bring the image/light spot landing on the PSD to within the range such that the PSD can sense/measure the centroid of sequentially sampled sub-wavefronts.

In one embodiment the dynamic wavefront/defocus offsetting device 139 comprises stacked liquid lenses. As illustrated in FIG. 1A, each liquid lens is represented by a cell comprising two different liquids such as water and oil. The interface between these two liquids can change from a concave shape to a convex shape depending on an activation signal such as an applied voltage value. Due to the fact the refractive index of the two liquids are different, when the interface changes its shape from a concave one to a convex one, the light focusing power of the liquid lens cell will thus change. As can be seen in FIG. 1A, assuming the interface is spherical, there is a limit to how concave or convex the interface can be shaped. By stacking three such liquid lens cells next to each other, the overall optical focusing power tuning range can thus be increased substantially.

Flood illumination light sources 140 are disposed near and surrounding the second optical window and ranging illumination light sources 142 are disposed within the wavefront sensor module 10 near and surrounding the first wavefront relay lens 110.

In this embodiment, the wavefront generation light beam is output from a superluminescent diode (SLD) 150 and guided to the wavefront relay path 105 through a second optical path 152 that includes a beam collimator 154 and two light beam folding mirrors 156 and 158. The wavefront generation light beam is launched to the patient eye through the PBS 130, the imaging beam splitter 160 and the first wavefront relay lens 110.

The imaging beam splitter 160 is disposed in the wavefront relay beam path and directs some light along a third optical path 161 through a lens 162 to an image sensor 164. The dichroic or short pass beam splitter 104 can be designed to also allow a portion of the visible and/or near infrared light outside the SLD spectrum range to be reflected/deflected so that a clear live image of the anterior of the patient eye can be captured by the image sensor 164. A light reflecting element 166 is also disposed along a fourth optical path 165 to intercept the SLD beam light reflected from the imaging beam splitter 160 so that a very small amount of the SLD beam can be sent to the image sensor 164 to be imaged to indicate if the SLD beam being launched to a patient eye is moved over time as compared to a reference point established when the wavefront sensor module is manufactured.

A band pass filter 168 can be arranged along the wavefront relay beam path anywhere between the imaging beam splitter 160 and the quadrant detector 108 to filter out any light outside the SLD spectrum to reduce background noise.

In FIG. 1, the lens shield 103 (which can be a spherical lens or a cylindrical lens or an aspheric lens or a free-form lens or a combination thereof) is used instead of a non-lens optical shield or window at the input port of the wavefront sensor module 10 to be attached to or integrated with an ophthalmic microscope 12. This lens is shared by the wavefront sensor and the surgical microscope to provide a changed working distance for a surgeon. Note that compared to a blank parallel optical window such as a piece of glass plate with proper coating, this lens will present a patient eye to the microscope and the wavefront sensor module with a virtual image of the eye. If the virtual image is to be positioned at the originally designed distance (for example, 175 mm from the objective lens of the microscope), the real eye needs to be positioned axially differently than at the originally designed distance due to the use of the lens. This is basically the mechanism that the working distance or space can be changed by using a lens rather than a non-lens optical shield.

FIG. 1B illustrates the operation of a negative lens to form a virtual image. As depicted in FIG. 1B, in an example embodiment the standard working distance of the optical microscope is 175 mm and the actual working distance to the patient eye is 185 mm. The negative shield lens 103 creates a virtual image of the patient eye at the standard working distance of 175 mm.

The operation of a negative shield lens 103 to create a virtual image will now be described with reference to FIG. 1C in which a stronger negative lens is placed closer to an object (the cornea of a patient eye). Light rays from the actual object 1000 are bent outwardly by the negative lens 103 so that the light rays appear to come and hence backwardly converge at the location of the virtual image 1002 of the object. Thus, although the position of the actual object 1000 has not changed, to an observer it appears that the object is now at the position of the virtual image.

There can be different reasons for the use of a negative lens as the shield instead of a non-lens optical window. One reason is that as the normal working space between the objective lens of an ophthalmic surgical microscope and the patient eye is now partially occupied by the wavefront sensor module 10. If the objective lens of the microscope is not changed there will be less working space left for a surgeon to maneuver his surgical tools without making contact with the wavefront sensor module. In other words, compared to a surgical microscope without the attached wavefront sensor module, it is more likely that a surgeon's hand(s) and tools may, during a surgery, make contact with the bottom surface of attached wavefront sensor module housing. Once a contact is made, the sterile field is broken and the surgeon will have to temporarily stop the surgery and change his gloves. This will consume surgery time and also cause frustration to the surgeon.

One solution to increase the working space between a microscope and a patient eye is to use a longer focal length objective lens in the microscope housing. However, normally, surgical microscope manufacturers only provide two or three difference focal length objective lenses and there is generally a relatively large difference in the focal length (normally a 25 mm step) between these objective lenses. An issue associated with a relatively large increase in working distance is that if a surgeon is relatively short and/or has become accustomed to an eye-to-hand working distance, a sudden increase by 25 mm in the distance can disrupt the surgeon's normal eye-hand coordination and cause frustration to the surgeon.

The benefit of using a negative lens as an optical shield to somewhat increase the working space to a surgeon preferred value is that the size of the increase can be better controlled (for example, by 5 mm or 10 mm or 15 mm instead of 25 mm). Optically, with a negative lens, the patient eye needs to be positioned further away from the microscope in order for a sharply focused virtual image of the eye to be formed at the originally designed distance (for example 175 mm). The power of the negative lens determines how far further away the patient eye needs to be positioned.

In most cases a surgical microscope is shared by a number of surgeons. Needs of different surgeons can be met by making the shield or shield lens field-replaceable. If the shield or shield lens is made field-replaceable then a different negative lens can be used for each surgeon to suit his/her particular preference. This can be beneficial for a surgeon since the change in the distance between a sharply focused patient eye and the surgeon's eye can now be only somewhat increased by a desired value that may improve ergonomics for a surgeon and at the same time reduce the chance of breaking the sterile field. Otherwise, a surgeon may be forced to change the objective lens of the microscope and typically tolerate a sudden large change of 25 mm (as the objective lens focal length from most microscope manufacturers is either 150 mm or 175 mm or 200 mm). By using a negative lens rather than a non-lens optical shield or window, an increase in the working distance from, for example, 5 mm to 15 mm can be provided to the surgeon to partially compensate the reduction in the working space as a result of the attachment of the wavefront sensor module to the microscope.

A second reason is that, due to the use of a tilted main beam splitter 104 and also the need to tilt the optical shield window 103 to prevent specular reflections of the microscope illumination light by the tilted optical shield window from entering the surgeon's eye, minor deterioration of the microscopic image quality presented to the surgeon may be introduced. One approach to address the issue is to tilt the optical shield window 103 in a perpendicular direction as compared to the main beam splitter 104 (i.e. if one looks at FIG. 1 from the left side, the optical shield window will appear with one side up and the other side down). However, this may not be enough or just right to completely cancel the minor deterioration to the microscopic image quality. As one aspect of the present disclosure, lens 103 can be made with a desired spherical and cylindrical power so that while the deterioration to the microscopic image quality is minimized, the change in the working distance is also optimized. Meanwhile, lens 103 can also be a pure spherical lens or an aspheric lens or a free-form lens or a combination of different lenses as long as it can bring benefits to the surgeon. In addition, as lens 103 can have a certain cylinder focusing power or component, the requirement to tilt an optical shield window perpendicular to that of the main beam splitter may no longer be necessary with lens 103. In other words, the lens 103 can be tilted in the same direction as the main beam splitter as long as the lens has the correct cylindrical power.

The same argument can also be made with respect to the wavefront sensor module. Note that in the currently disclosed optical configuration of FIG. 1, the wavefront generation light beam (from a superluminescent diode or SLD 150 through a beam collimator 154 and two light beam folding mirrors 156 and 158) is launched to the patient eye through two beam splitters 130 and 160 and the first wavefront relay lens 110. While the two beam splitters 130 and 160 are tilted substantially (for example at about 45 degrees to serve the function of light beam splitting and combining), the first wavefront relay lens 110 is not. For wavefront relay purpose, the first wavefront relay lens 110 should ideally be positioned with its central front and back surfaces normal to the optical axis 105. However, since the wavefront generation beam is launched from behind the first wavefront relay lens 110, if the lens 110 is not tilted, specular reflections of the wavefront generation beam by the front and back surface of the lens 110 can be channeled to the wavefront sensing detection path and produce optical noise that can degrade the performance of the wavefront sensor. In order to prevent this from happening, there is thus a need to tilt the lens 110. The lens 110 can be tilted as shown in FIG. 1, but can also be tilted orthogonal to what is shown in FIG. 1, i.e. the tilting can be observed as one side up (or left displaced) and the other side down (or right displaced) when one looks from the top of FIG. 1. It can also be tilted in other directions. As such, there will be some inherent optical system aberrations such as astigmatism that is introduced to the wavefront sensor as a result of the tilting of the first wavefront relay lens 110. While calibration, software and data processing can generally be employed to cancel the inherent system aberrations such as astigmatism or other higher order wavefront aberrations, it is always preferred to keep any inherent optical system aberrations such as astigmatism to a minimum. As one aspect of the present disclosure, the tilting angle and the thickness of the first wavefront relay lens 110 are properly selected such that its specular reflections are not channeled to the wavefront detection path. As another aspect of the present disclosure, the tilting of the first wavefront relay lens 110 can be in the orthogonal direction as compared to that of the shield or lens 103 so that partial or full cancellation of optical system aberrations such as astigmatism for the wavefront relay beam path can be achieved. In addition , it may be possible to design the first wavefront relay lens 110 such that the induced inherent optical system aberrations such as astigmatism as a result of the tilting is approximately the same as that introduced to the microscopic view by the main beam splitter (Note in FIG. 1 that the tilting direction of the first wavefront relay lens 110 is the same as that of the main beam splitter, and as the wavefront relay beam does not pass through the main beam splitter which is a front surface reflector for the wavefront relay beam, the main beam splitter does not introduce optical system aberration such as astigmatism to the wavefront relay beam). As such, lens 103 can be designed with an appropriate sphere and cylinder power to change the working distance by a desired amount and also to conveniently cancel inherent system aberration such as astigmatism introduced to the wavefront relay beam path.

Note again that although the tilting of the first wavefront relay lens 110 as shown in FIG. 1 is in the same direction as that of the main beam splitter, this does not need to be the case. In fact, by tilting lens 103 in a direction perpendicular to that of the main beam splitter 104 and the first wavefront relay lens 110, some degree of cancelation of optical system aberration such as astigmatism can already be achieved.

In addition to tilting the lens 110, as another aspect of the present disclosure, the lens 110 can be also be positioned slightly off center such that specular reflections of the SLD beam from the front and back surfaces (or even an intermediate surface if the lens is a doublet) are deflected outside the wavefront detection path, i.e. SLD specular reflections from the first wavefront relay lens 110 are spatially filtered and hence will not reach the wavefront sensor detector. The slight tilting or off-center positioning of the lens 110 can also be in other direction depending on if there are other optical system aberrations such as astigmatism created downstream along the light beam path in either the microscope or the wavefront sensor module. For example, if there is or are other optical component(s) further downstream of the wavefront beam relay path that can also produce inherent optical system aberrations such as astigmatism, the first wavefront relay lens 110 can be designed with a desired thickness and tilted or off-center-positioned to partially or fully cancel those additional astigmatisms (in addition to what was discussed before).

One aspect of the present disclosure is the use of the transmissive wavefront compensator or offsetting device 139, comprising at least one focus variable lens, arranged at the intermediate wavefront image plane to compensate at least the sphere component (either partially or fully) of the relayed wavefront there. The transmissive wavefront compensator or offsetting device 139 can be two or more focus variable lenses stacked next to each other. FIG. 1A shows an example embodiment in which three liquid-lens-based focus variable lenses are stacked together or next to one another to offer an increased focusing power tuning range. Note that the compensation should not be limited to the sphere component only but can be extended to other wavefront components such as cylinder and coma as long as the focus variable lenses have the capability.

A MEMS scan mirror 134 is disposed at the second Fourier transform plane of the 8-F wavefront relay to angularly scan the wavefront relay beam so that the relayed wavefront at the final wavefront image plane can be transversely shifted relative to the wavefront sampling aperture 106. The wavefront sampling aperture 106 can be a fixed size aperture or an active variable aperture. One aspect of the present disclosure is the coating of the wavefront sampling aperture with an extremely light absorptive black layer such as an Acktar black coating so that those portions of light that are blocked by the aperture are substantially absorbed by the aperture coating and hence are not channeled or minimally channeled to the wavefront sensing detector. The black coating layer is preferably deposited or formed at least on the front side of the aperture facing the incoming wavefront relay beam, but it can also be made on both sides. The blackness or strong absorbance of light can also be achieved by using a really black absorptive material to make the aperture.

As another aspect of the present disclosure, a properly designed diffuser 109 is arranged after a sub-wavefront focusing lens 107 and an aperture 106. The sub-wavefront focusing lens focuses sequentially sampled sub-wavefront through the aperture to the diffuser which diffuses the sampled sub-wavefront beamlet to slightly increase the light spot size when the sampled beamlet reaches the position sensing detector 108. The diffusing effect will make sure that even when the sampled sub-wavefront has a curvature that can cause the sampled beamlet to be otherwise sharply focused by the sub-wavefront focusing lens to a tiny diffraction limited spot, when such beamlet passes through the diffuser, the beamlet will expand and arrive at the position sensing detector with a larger spot size that will be shared by different photosensitive pixels (if the position sensing detector is a 2D detector array) or areas (if the position sensing detector is a quadrant detector). This is extremely beneficial when the position sensing detector is a quadrant detector simply because in such a case if the light spot is too small, it can easily become not being shared by all the quadrants. In a case where the sub-wavefront focusing lens has an effective focal lens of about 3 mm to 5 mm, a diffuser with a diffusing angle from about 5 degrees to 20 degrees, or more preferable from about 10 degrees to 15 degrees can be arranged at or near the back focal plane of the sub-wavefront focal lens and act as the window of the quadrant detector. In one aspect, the wavefront sensing detector is directly made with a diffuser window. In another aspect, a separate diffuser with its diffusing surface facing away from the wavefront sensing detector is optically bonded to the window of the wavefront sensing detector.

An electronics system is connected to the SLD 150, the wavefront shifting MEMS scan mirror 134, and the light spot position sensing detector (PSD) 108, to pulse the SLD, scan the MEMS mirror and collect the signal from the PSD in synchronization such that lock-in detection can be employed to filter out any temporal optical noise outside the SLD pulsing frequency band.

In addition, the electronics system is also connected to the transmissive wavefront compensator or offsetting device 139 such as a stack of focus variable lenses as shown in FIG. 1A with software control. One aspect of the present disclosure is to servo the stack of focus variable lenses 139 so that a targeted compensation of the sphere component of the relayed wavefront at the focus variable lenses location is achieved. In general, the focusing power tuning range (in diopter) of a variable focal length lens is inversely correlated to the transverse size or diameter of the lens, i.e. a larger diameter lens will have a smaller focusing power tuning range and a smaller diameter lens will have a larger focusing power tuning range. In an attempt to make the optical design compact so that a wavefront sensor module can be attached to or integrated with an ophthalmic surgical microscope and, at the same time, to use a commercially available small beam scanner to fully intercept a wavefront relay beam over a large diopter measurement range, the optical configuration as shown in FIG. 1 is designed to make the relayed wavefront beam transversely de-magnified at certain axial locations where the beam will be fully intercepted by a variable focal length lens and a beam scanner. Typically, the variable focal length lens is located at a transversely de-magnified optical conjugate plane of the object wavefront plane and the beam scanner is located at a Fourier transform plane. However, the substantial de-magnification in the transverse dimension of the relayed wavefront beam at the intermediate wavefront image plane where the variable focal length lens is located also means a significant magnification or amplification in the vergence or the diopter value variation of the relayed wavefront there. The diopter variation range of the wavefront relayed to the intermediate wavefront image plane at the location of variable wavefront compensator or offsetting device 139 will be increased substantially as compared to that at the initial wavefront object plane (i.e. the corneal or pupillary plane of the patient eye).

Due to the fact that commercially available variable focal length lens has a limited focusing power tuning range, to overcome the limitation of nulling or partially compensating the sphere component over a large object wavefront diopter range while still maintaining the compactness of the optical configuration, as one aspect of the present invention, two or more variable focal length lenses are stacked together and arranged at or near an intermediate wavefront image plane conjugate to both the initial wavefront object plane and the final wavefront image plane. By stacking two or more focal length variable lenses, enough sphere compensation can be realized to cover the needed diopter range that is required for a typical cataract surgery, i.e. to cover the typical refraction change of a patient eye from its phakic state to aphakic state and then to its pseudo-phakic state.

As another embodiment, the wavefront compensator or offsetting device 139 can be a variable lens that can change both the sphere and the cylinder or astigmatism component of a relayed wavefront to null or compensate (either partially or fully) the sphere and/or cylinder/astigmatism component of the object wavefront from a patient eye to further improve the accuracy and precision of wavefront measurement over a large diopter range. It should be noted that such a variable lens is already commercially available from Varioptic and can be directly employed in the presently disclosed design.

As still another embodiment, the wavefront compensator or offsetting device 139 can be a combination of one cylinder or astigmatism variable lens and at least one focus variable lens. The combination can be used to null or compensate (either partially or fully) the cylinder/astigmatism and/or the sphere component of the object wavefront from a patient eye to further improve the accuracy and precision of wavefront measurement over a large diopter range.

In addition, to further minimize optical noise that can be channeled to the wavefront sensing detector, other aspects of the invention are also disclosed here. Note that optical noises outside the SLD spectrum band can be filtered out through spectral filtering using optical band pass filters. Optical noises not having the same temporal pulsing frequency as the SLD is modulated at can be filtered out using electronic lock-in detection technique. The remaining optical noises that have the same spectral range as that of SLD and are also pulsed at the same frequency of the SLD are typically specular reflections of the SLD beam from any optical interface other than from the retina of a human eye. Although polarization extinction means which is realized as shown in FIG. 1 with the use of the PBS 130 are generally adopted to remove those SLD specular reflection noises having the same polarization as that of the incident SLD beam, the limited extinction ratio of a polarization beam splitter (PBS) and the fact that SLD specular reflection can hit the PBS at angles other than the designed incidence angle mean that there can still be optical noise from SLD specular reflections that may be channeled to the wavefront sensing detector. We already discussed one source of SLD beam specular reflection that comes from the first wavefront relay lens 110 as the SLD beam is launched from behind the lens 110. Another source of SLD beam specular reflection is from the cornea and the natural lens or the implanted artificial lens of a biological eye. These reflections can also have a small amount of polarization component that is in the orthogonal polarization direction of the incident SLD beam, i.e. this component cannot be filtered out by the PBS through polarization extinction means.

As other aspects of the present disclosure, further spatial filtering means of these optical noises are employed. In one embodiment, the SLD specular reflections from the cornea and/or the lens of the eye (either natural or artificial) are relayed to within a small volume around the wavefront image plane where the wavefront sample aperture 106 is located and the scanning angle range of the wavefront beam scanner (134) is properly selected so that these SLD specular reflections from the cornea or ocular lens (either natural or artificial) are effectively blocked by the wavefront sampling aperture 106. In another embodiment, a mega aperture 136 is disposed at a first Fourier transform plane of the 8-F wavefront relay so that light rays coming out from a patient eye that are outside a certain light acceptance cone range are blocked by the mega aperture. The mega aperture will also prevent these unwanted light rays from hitting the non-movable portion of the beam scanner located at the second Fourier transform plane of the 8-F relay.

In addition to the superluminescent diode (SLD) beam launching path and the folded wavefront relay beam path, a few more optical paths are also shown in FIG. 1, one for flood illuminating the eye for live patient eye imaging, one for sending distance measuring or ranging light beams to the patient eye, one for various imaging with an image sensor, one for internally creating a reference wavefront inside the wavefront sensor module for internal calibration and verification checking of the performance of the wavefront sensor module, one for directing a very small amount of the SLD beam to an image sensor for internal calibration and verification checking of the SLD beam being sent to a patient eye, and one for directing a fixation target to the patient eye.

Several flood illumination light sources such as near infrared LEDs 140 operating at a near infrared wavelength or spectral band different from that of the SLD are arranged around lens 103 to send light to flood illuminate the patient eye. Two or more distance measuring ranging light beams (which preferably operates at the same near infrared wavelength or spectral band as the flood lights) are preferably arranged inside the wavefront sensor module to send ranging light beams with crossing light propagation angles to the patient eye so that optical triangulation can be used to measure the distance of the eye relative to the wavefront sensor module. An imaging beam splitter 160 directs at least some of the imaging light returned from the eye through an imaging lens 162 to an image sensor 164, such as a 2D pixel array CCD/CMOS sensor. The image sensor 164 can be a monochrome black/white CMOS/CCD image sensor connected to the electronics system. The imaging beam splitter 160 can be properly coated to reflect only the flood and ranging illumination spectral band of light to the image sensor and to transmit as much as possible the SLD spectral range of light. In this way, if there is no illumination from the surgical microscope or if the illumination light from the surgical microscope has been filtered to only allow visible light to reach the eye, the contrast of the eye image as captured by the image sensor 164 can be kept to within a desired range. To further remove optical noise from unwanted lights such as the illumination light from the microscope, an additional optical band pass filter can be arranged right in front of the image sensor to only allow light within desired spectral range(s) such as the flood illumination and ranging illumination spectral range to pass through. The image sensor 164 provides a coplanar video or static image of a patient eye and can be focused to image the anterior of the eye.

The flood illumination lights 140 can be a ring or multiple rings of LEDs (or arrays) arranged encircling around the input port of the wavefront sensor module. In addition to simply providing flood illumination, the flood illumination LEDs 140 can be used to create specular reflection light spot images from the optical interfaces of the cornea and/or the eye lens (natural or artificial) so that Purkinje images of the LEDs 140 can be captured by the live image sensor (164) and displayed live to the surgeon to help the surgeon in aligning the patient eye to the wavefront sensor module. As one aspect of the present disclosure, these live Purkinje images are used to show in real time how well a patient eye is aligned in term of the eye position in x-y-z and also in terms of the tip and tilt angle of the eye relative to the optical axis of the wavefront sensor module.

As another aspect of the present disclosure, the ranging illumination light sources 142 can be selectively turned on and off, and projected onto the white of the eye in synchronization with the progressive or global shutter of the image sensor 164 to sequentially create or not create light spots for certain frames of live eye image for the image sensor 164 to capture. As a result, frame subtraction can be done before centroiding the light spots to realize better eye distance measurement using the principle of optical triangulation. Note that with optical triangulation, the change in the centroid position of the imaged light spots can be processed to figure out the eye distance.

Furthermore, to simplify the self-calibration checking or verification of the wavefront sensor, a dichroic beam splitter/mirror 132 is combined with a reference wavefront generating light source 138 such as a light emitting diode (LED) disposed inside the wavefront sensor module to generate a reference wavefront for the wavefront sensor to measure. Preferably, the reference wavefront generating light source 138 is located somewhere near the front focal plane of the second wavefront relay lens 112 so that the reference wavefront generated for the wavefront sensor to measure is approximately like an emmetropic wavefront from a patient eye. However, the reference wavefront generating light source can also be located somewhere else as long as it can generate a reference wavefront inside the wavefront sensor module. In addition, the self-generated reference wavefront beam can also be made to travel through the wavefront compensator or offsetting device 139 such as one or a stack of focus and/or astigmatism variable lens(es) such that by changing the focus and/or the astigmatism of the focus and/or astigmatism variable lens(es), the response or transfer function of the wavefront sensor over a relatively large dioptric variation range can be checked. In addition, as for the SLD source case, the reference wavefront generating light source 138 can be pulsed (with the SLD source turned off) in synchronization with the scanning of the wavefront shifting MEMS scan mirror 134, and operation of the light spot position sensing detector (PSD) 108 such that lock-in detection can be employed to filter out any temporal optical noise outside the reference wavefront generating light source pulsing frequency band.

As another embodiment, a very small fraction of the SLD beam launched to the patient eye from inside the wavefront sensor module is channeled to the image sensor inside the wavefront sensor module so that the SLD beam pointing direction can be checked over time to ensure that the beam is not moved. In FIG. 1, this beam path is shown by the dotted line 165. Note that when the SLD beam launched from behind the PBS 130 hits the imaging beam splitter 160, a very tiny fraction of the SLD beam will be reflected by the imaging beam splitter/mirror 160. In the present disclosure, a light reflecting element 166 is disposed along the dotted line 165 to reflect a portion of the side reflected SLD beam by the imaging beam splitter/mirror 160 and direct it to the image sensor 164 through the imaging lens 162. The light reflecting element 166 can be a neutral density filter or a mirror. In this respect, the SLD and the flood illumination (as well as the ranging) LEDs can be selected with a slight spectral overlap so that when a band pass filter is disposed right in front of the image sensor 164, it will still allow most of the flood illumination and ranging lights to pass through and also allow a very small amount of the SLD light to pass through.

As the SLD beam being directed along the dotted line 165 is polarized due to the fact it passed through the PBS 130, as one aspect of the present disclosure, a polarizer (not shown in FIG. 1) can be placed in front of the light reflecting element 166 to control the amount of SLD light that can be directed to the image sensor 164. The polarizer can have a tilt angle to direct any specular reflection from the polarizer away from entering the image sensor 164. The portion of light that passes through the polarizer, reflected back from the light reflecting element 166 and passes again through the polarizer a second time can be controlled in terms of its optical power or energy by rotating the polarizer so that on the image sensor, a faint SLD light spot can be created. This faint reference SLD light spot will overlap on the live eye image but as long as its relative brightness is properly controlled and its location is outside the central region of most interest to a surgeon, it is generally not a problem to a surgeon. Again, for the SLD beam position calibration checking, the SLD beam can be synchronized with the image sensor frames and “bright” and “dark” frames can be captured to enable frame subtraction to better determine the centroid position of this reference SLD beam position on the image sensor 164.

As one embodiment of the present disclosure, the reference wavefront generated readings (which can be diopter values as well as other signals such as wavefront signal strength) measured by the wavefront sensor with or without activating the transmissive wavefront compensator or offsetting device 139 and the reference SLD beam position as captured by the image sensor 164 can be stored as reference at the time when the device is manufactured, and later during any patient session when the device is to be used for a patient, a self-calibration checking can be done to compare the readings with the stored reference values. If the difference is greater than a predefined threshold, a message can be displayed to the surgeon to indicate that the device is out of its calibration or expected performance and needs to be recalibrated or serviced.

Further, a fixation target 170 can be arranged below and near the center of the objective lens of the microscope to guide the patient in aligning his/her eye to the optical axis of the wavefront sensor module. The fixation light source can be a red or green or yellow (or any color) light emitting diode (LED). One aspect of the present disclosure is to blink the fixation light so that the surgeon can instruct the patient to look at the blinking light to differentiate the fixation light from the microscope illumination lights.

In addition to hardware improvements, software and algorithm related improvements are also disclosed here. One embodiment is to automatically detect the state of the patient eye, especially when a patient eye is transformed from a phakic state to an aphakic state and then to a pseudo-phakic state. The transition can be detected using both the wavefront signal(s) and the live image of the eye.

In the phakic state, as the eye to be operated on generally has some degree of cataract that scatters light, the wavefront signal strength is generally weaker than that when the eye is in its aphakic state with the cataract lens removed. Therefore, one aspect of the present disclosure is to measure and compare the wavefront signal strength to determine if a patient eye is transformed from a phakic state to an aphakic state. Another aspect is to combine wavefront signal (such as its strength) detection with image processing of the live eye image to better determine the state of the eye. One approach is to only compare wavefront sensing signal strength when the live image of the eye shows that the eye is optically aligned with the wavefront sensor module. Another approach is to also detect Purkinje images of the flood lights specularly reflected from the natural lens of the eye. These third and fourth Purkinje images from the natural cataract lens will be weak as compared to those produced by an artificial lens or IOL (intra-ocular lens) at the pseudo-phakic stage. Note also that these third and fourth Purkinje images will disappear when the natural lens is removed at the aphakic stage. Although automatic detection of the eye state is preferred, there can be rare cases when the system fails to detect. In such a case, one aspect of the present invention is to provide a surgeon with a manual way of designating the state of a patient eye.

Previously, we have disclosed using various qualifiers to filter out bad wavefront data and pass only good “qualified” wavefront data for display to a surgeon. In this disclosure, as one embodiment, for each state of the patient eye, a further qualification is performed by running and displaying time-averaged refraction or wavefront of the eye under operation that is most stable.

Because one requirement for a real time wavefront sensor is to provide calculated IOL (intra-ocular lens) power using live measured aphakic refraction, and a challenge is to figure out, while the eye is dynamically moving during an aphakic refraction measurement, which of the many real time measured aphakic refraction values should be used for the calculation. One embodiment of the present disclosure is to automatically and/or continuously feed those most stable time-averaged wavefront or refraction data obtained at the aphakic state of a patient eye into an IOL algorithm calculator. Still another embodiment is to display the continuous result from the IOL algorithm calculator as a predicted IOL based on target refraction that is defaulted to emmetropia but can be manually entered pre-operatively if the target dioptric value is different from zero (such as -1.0 diopter).

When the eye state is changed from an aphakic state to a pseudo-phakic state, in addition to a slight reduction in the wavefront signal strength as an IOL will remove a small portion of wavefront generation SLD beam and also the retina returned wavefront signal, another major detectable signal is the IOL reflected Purkinje images. This is because an implanted IOL generally has a high refractive index than that of the natural lens, and as a result, the IOL induced Purkinje images from the front and back surface of the IOL is generally much brighter than those of the natural lens of a patient eye. Note that when the eye is in its aphakic state, there are no third and fourth Purkinje images from either a natural lens or an implanted lens as there is no lens in the eye bag or capsule.

As one aspect of the present disclosure, the third and/or fourth Purkinje images produced by an implanted IOL is used as a criterion to determine if an eye is in its pseudo-phakic state. In addition, as another embodiment, the IOL induced Purkinje images can also be combined with a somewhat reduced wavefront signal strength when the eye is optically aligned with the wavefront sensor module to determine if the eye is in its pseudo-phakic state. Still another embodiment is to automatically turn off the IOL calculator once the system detects that the surgical eye state is changed to its pseudo-phakic state.

Furthermore, as another aspect of the present disclosure, a bookmark/tickmark can be automatically or manually generated to record the time-averaged value of the IOL prediction as desired.

Although various embodiments that incorporate the teachings of the present invention have been shown and described in detail herein, those skilled in the art can readily devise many other varied embodiments that still incorporate these teachings. Further, all patents, patent applications, academic articles, and other such references identified in the present disclosure are herein incorporated by reference.

What is claimed is:

1. A wavefront sensor module configured to be attached to or integrated with an ophthalmic instrument for eye examination and/or vision correction procedures where the standard axial working distance of an objective lens of the ophthalmic instrument is the distance between a light input window of the ophthalmic instrument and a subject eye along the optical axis of the objective lens, with the wavefront sensor module comprising oppositely disposed first and second surfaces and an interior, with a module thickness substantially equal to the separation between the first and second surfaces, with the first surface having a first optical window configured to pass light between the interior of the wavefront sensor module and a subject eye and with the second surface having a second optical window disposed substantially adjacent to the objective lens and configured to pass light between the interior of the wavefront sensor module and the ophthalmic instrument, where the first and second optical windows are aligned to form an optical path along the optical axis of the objective lens to pass light between a subject eye and the ophthalmic instrument via the interior of the wavefront sensor module, where the actual axial working distance is greater than the standard axial working distance of the ophthalmic instrument to substantially or partially compensate the module thickness and with the wavefront sensor module comprising:
a negative front lens disposed substantially at the first optical window of the wavefront sensor module, with the negative front lens configured to outwardly bend light rays returning from the patient eye so that the negative lens forms a virtual image at the standard working distance of the ophthalmic instrument;
a beam splitter/combiner disposed along the optical path to intercept light transmitted by the negative lens, with the beam splitter/combiner configured to transmit at least a portion of the light returned from the subject eye meant for the ophthalmic instrument, and to reflect at least a wavefront beam returned from the subject eye to the interior of the wavefront sensor module.

2. The wavefront sensor module of claim 1 further comprising:
a wavefront relay optical system, including first and second lenses having optical axes, disposed in the interior of the wavefront sensor module and configured to relay the wavefront beam reflected from the beam splitter/combiner along a wavefront relay optical path internal to the wavefront sensor module and wherein the first lens of the wavefront relay is oriented so that the optical axis of the first lens is tilted by a small angle from the wavefront relay optical path so that specular reflection by the first lens from an internal light source is not directed along the wavefront relay optical path to arrive at the wavefront sensor detector.

3. The wavefront sensor module of claim 1 further comprising:
a wavefront relay optical system, including first and second lenses, disposed in the interior of the wavefront sensor module and configured to relay the wavefront beam reflected from the beam splitter/combiner along a wavefront relay optical path internal to the wavefront sensor module; and
a wavefront sampling aperture disposed at an image plane of the wavefront relay optical system, with the wavefront sampling aperture having a highly absorptive coating to absorb a portion of the wavefront beam incident on the wavefront sampling aperture.

4. The wavefront sensor module of claim 1 further comprising:
a wavefront relay optical system, including first and second lenses, disposed in the interior of the wavefront sensor module and configured to relay the wavefront beam reflected from the beam splitter/combiner along a wavefront relay optical path internal to the wavefront sensor module; and
a cone angle limiting aperture disposed at a Fourier transform plane of the wavefront relay optical system configured to limit a cone angle of light rays in the wavefront beam and limit the diopter range of the wavefront beam.

5. A wavefront sensor module configured to be attached to or integrated with an ophthalmic instrument for eye examination and/or vision correction procedures comprising:
a wavefront relay optical system, including first and second lenses having optical axes, disposed in the interior of the wavefront sensor module and configured to relay the wavefront beam reflected from the beam splitter/combiner along a wavefront relay optical path internal to the wavefront sensor module; and
at least two stacked dynamic focus variable lenses at or near a conjugate plane of an object wavefront within the wavefront relay optical system to substantially or partially null or compensate (either partially or fully) the sphere component of the object wavefront from a patient eye to improve the accuracy and precision of wavefront measurement over a large diopter range.

6. A wavefront sensor module configured to be attached to or integrated with an ophthalmic instrument for eye examination and/or vision correction procedures comprising:
a wavefront relay optical system, including first and second lenses, disposed in the interior of the wavefront sensor module and configured to relay a wavefront beam reflected from a beam splitter/combiner along a wavefront relay optical path internal to the wavefront sensor module; and
a wavefront sampling aperture disposed at an image plane of the wavefront relay optical system, with the wavefront sampling aperture having a highly absorptive coating to absorb a portion of the wavefront beam incident on the wavefront sampling aperture.
